(19) United States
(12) Patent Application Publication
(10) Pub. No.: US 2007/0077220 A1
(43) Pub. Date: Apr. 5, 2007

(54) Stable ascorbic acid compositions

(76) Inventors: Jose E. Ramirez, Trumbull, CT (US); Judy Hattendorf, Marina Del Ray, CA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/541,090

(22) Filed: Sep. 28, 2006

Related U.S. Application Data
(60) Provisional application No. 60/722,511, filed on Sep. 30, 2005.

(51) Int. Cl. A61K 8/49; A61K 31/70; A61K 31/045; A61K 31/375

(52) U.S. Cl. 424/70.1; 514/23; 514/474; 514/738

(57) ABSTRACT

Compositions include vitamin C, water and a reducing sugar, result in increased stability of vitamin C. Further the addition of alcohol to aqueous vitamin C compositions increased the stability of vitamin C. Moreover, aqueous vitamin C compositions having surfactant included therein enhance the absorption of vitamin C into skin. Methods of using the stable vitamin C compositions are also described.

### CROSS REFERENCE TO RELATED APPLICATION

This Application claims priority benefit of U.S. Provisional Application No. 60/722,511 filed Sep. 30, 2005 the entire disclosure of which is incorporated herein by this reference.

### BACKGROUND

1. Technical Field

The present disclosure relates to ascorbic acid compositions exhibiting markedly improved chemical stability and methods of making them. Furthermore, topical compositions (e.g., pharmaceutical and/or cosmetic based products) containing L-ascorbic acid stabilized in aqueous solution are described along with topical application thereof to skin.

2. Background of Related Art

L-ascorbic acid is a water-soluble, antioxidant vitamin used in many products. For example, L-ascorbic acid is used in pharmaceutical and cosmetic products as an active ingredient for therapeutic treatment. L-ascorbic acid has therapeutic, corrective and/or cosmetic significance in that it is important in forming collagen, cartilage, muscle, and blood vessels. L-ascorbic acid also aids in the absorption of iron, and helps maintain capillaries, bones, and teeth. L-ascorbic acid further promotes healthy cell development, proper calcium absorption, normal tissue growth and repair. Moreover, L-ascorbic acid prevents blood clotting and bruising, while strengthening the walls of the capillaries. L-ascorbic acid is also important for healthy gums, protecting against infection, assisting with the clearing up of infections, reduction of cholesterol levels, lowering of high blood pressure and preventing arteriosclerosis. Consequently, deficiencies of ascorbic acid leads to problems such as, among other things, scurvy, hemorrhages under the skin, bruising, poor wound healing, soft and spongy bleeding gums, loose teeth, edema, weakness, lack of energy, poor digestion, painful joints, bronchial infection and colds.

L-ascorbic acid is chemically defined as an alpha-ketolactone with the following chemical structure: 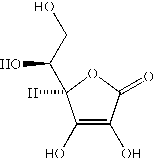
The number 2 and 3 carbons are double-bonded and contain acid-ionizable hydrogen in water (pK=4.2). Ascorbic acid is moderately strong reducing agent. Unfortunately, these properties lead to instability in the ascorbic acid structure which is burdensome to formulators attempting to prepare ascorbic acid solutions such as aqueous solutions. In particular, at higher pH's, the ascorbic acid increasingly becomes the unstable ascorbate anion (the conjugate base of ascorbic acid), which is susceptible to degradation.

The instability of ascorbic acid may be caused by a number of factors including stereochemical strain. For example, when the 2-hydroxy group ionizes, it places two negative charges in close proximity which favors ring disruption. Furthermore, oxidative degeneration likely promotes instability due to the ascorbate anion's propensity to act as a reductant, thus the molecule is prone to breaking down to form species such as L-threonic acid and oxalic acid. Such breakdowns can be catalyzed by the presence of a transition metal. Degradation may also occur due to a bulk water attack. Thus at lower ascorbate concentrations or ionic strength, water can react with ascorbic acid and degrade the molecule.

Various attempts have been made to produce stable solutions of L-ascorbic acid and its salts, but have been met with poor success. For example, U.S. Pat. No. 2,187,467 (the entire disclosure of which is incorporated herein by this reference) discloses aqueous solutions of ascorbic acid stabilized by the addition of salts of alkaline earth metals, ammonium, and soluble salt of a sulfite acid. However, this patent states that the stabilization was not achieved with the acid itself.

Other attempts at obtaining stable ascorbic acid compositions have been obtained by using expensive reagents and have also yielded a product with less desirable stability properties than ascorbic acid in its unmodified form.

The instability of L-ascorbic acid leads to reduced efficacy due to loss of active plus a variety of disadvantages, including short shelf lives, required expiration dating, higher product costs, and special storage considerations. Accordingly, compositions in which ascorbic acid is stable are desirable.

### SUMMARY

Aqueous compositions containing vitamin C, and a reducing sugar exhibit excellent stability. Such compositions containing vitamin C, water and a reducing sugar can be formulated to provide products with satisfactory shelf life. The markedly improved stability also leads to product forms that were previously not obtainable, such as, for example, aqueous vitamin C, and solutions where vitamin C remains stable at pH's of 2-5, and/or below 5. It has also been found that applying stable compositions of vitamin C having a pH below 5 increases the percutaneous absorption of vitamin C in skin. Moreover, the inclusion of surfactant in stable vitamin C solutions has been found to increase the percutaneous absorption of vitamin C in skin. Alcohols used in some formulations also enhance stability.

In embodiments, the present disclosure relates to a method of increasing absorption of vitamin C in skin by topically applying to skin a composition including vitamin C, water, surfactant, and a reducing sugar.

In embodiments, the present disclosure further relates to a method of improving the appearance of skin including the steps of: (a) stabilizing ascorbic acid in a solution which includes water, a reducing sugar, and salt, wherein at least about 0.1% of the total weight of the solution is reducing sugar; and (b) topically applying the solution to the area of skin to be affected such that the ascorbic acid is absorbed by the skin.

In embodiments, the present disclosure relates to a composition including vitamin C in an amount greater than 15% by weight of the total composition, a salt such as acid addition salt, base addition salt, metal salts, alkali metal salts, alkaline earth metal salts, ammonium salts, amine addition salts, amino acid addition salts, and/or combinations thereof, water in an amount greater than 40% by weight of the total composition, a secondary reducing agent, and reducing sugar such as mannitol, sorbitol, xylitol, maltitol lactitol, and/or combinations thereof.

### DESCRIPTION OF THE PREFERRED EMBODIMENTS

Stable vitamin C compositions for skin care in accordance with this disclosure are formulated in a manner which enables the vitamin C to remain stable when mixed with water. Compositions in accordance with this disclosure are effective in enhancing the penetration of vitamin C in the skin.

The compositions of the present disclosure contain vitamin C and a unique mixture of ingredients in an aqueous solution. The term “vitamin C” as used herein applies to substances that possess antiscorbutic activity. Such substances include, for example, L-ascorbic acid, commonly called ascorbic acid, salts of L-ascorbic acid, L-dehydroascorbic acid and salts of L-dehydroasorbic acid. L-ascorbic acid is a well known compound of general formula:

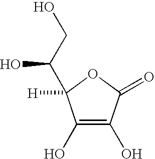

Suitable salt forms of vitamin C include any salt formed from the neutralization of ascorbic acid. Non-limiting examples include sodium ascorbate formed by the neutralization ascorbic acid with sodium to form L-ascorbic acid-monosodium salt. Other non-limiting examples of useful forms include calcium ascorbate, magnesium ascorbate, potassium ascorbate, manganese ascorbate, zinc ascorbate, molybdenum ascorbate, chromium ascorbate, and combinations thereof.

The vitamin C may be present in amounts that provide a benefit to the skin of a user. In embodiments, vitamin C is present in an amount sufficient to promote therapeutic, corrective and/or cosmetic treatment of a user's skin. The vitamin C present may be in acidic form, salt form, or mixtures thereof. As an illustrative example, vitamin C in amounts of about 5% to about 40% by weight of the total composition may be suitable. In embodiments, vitamin C is present in an amount of about 15% to about 25% by weight of the total composition, and in some embodiments in amounts of about 18% to about 22% by weight of the total composition.

The aqueous solution may further include water, one or more reducing sugars, one or more antimicrobial preservatives, one or more salts, one or more reducing agents, one or more surfactants, one or more alcohols, one or more conditioners such as Na Hyalurate, fragrance, and combinations thereof.

Suitable water for use in compositions in accordance with the present disclosure include tap water and/or purified water such as for example de-ionized water or USP water. As a non-limiting example, water may be present in compositions in accordance with the present disclosure in an amount of about 40% to about 96% by weight of the total composition. In embodiments, water may be present in amounts of greater than 40%, 50%, 60%, 70%, 80%, or 90% by weight of the total composition. In some embodiments, water is present in an amount greater than 40% by weight of the total composition and vitamin C is present in an amount greater than 15% by weight of the total composition.

Suitable reducing sugars for use in compositions in accordance with the present disclosure include sugars with a ketone or aldehyde group such that the sugar is capable of acting as a reducing agent. Non-limiting examples of reducing sugars include mannitol, sorbitol, xylitol, maltitol, lactitol, and/or combinations thereof. In vitamin C and reducing sugar compositions, it is believed that the reducing sugar oxidizes first and delays the start of any oxidation of the vitamin C so that excessive oxidation in water is delayed or totally avoided. Typically, the reducing sugar is present in an amount of about 0.1% to about 10.0% by weight of the total composition. In embodiments, reducing sugar is present in amounts of about 0.5% to about 5.0% by weight of the total composition.

Optionally, the reducing sugars may be mixed with water to form a reducing sugar solution that can be used to formulate a stable vitamin C composition in accordance with this disclosure. The reducing sugar solution may contain, for example, reducing sugar in an amount of about 1% and about 99% by weight of the total reducing sugar solution. In embodiments, the reducing sugar solution may contain about 70% by weight of the total reducing sugar solution. The amount of reducing sugar solution used to formulate the stable vitamin C composition will depend upon a number of facts including the concentration of reducing sugar in the solution. Typically, however, for a 70% solution, the reducing sugar solution may be added to the composition in an amount of about 0.25% to about 10.0% by weight of the total composition. In embodiments, such reducing sugar solution is admixed in an amount of about 1% to about 5% by weight of the total composition.

In embodiments in accordance with the present disclosure, sorbitol is used as a reducing sugar. Sorbitol, also known as glucitol is a sugar alcohol having the general formula:

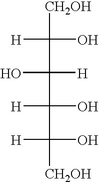

Sorbitol is a sugar alcohol (also known as polyol, polyhydric alcohol, or polyalcohol) which is a hydrogenated form of carbohydrate, whose carbonyl group (aldehyde or ketone, reducing sugar) has been reduced to a primary or secondary hydroxyl group. In one embodiment, sorbitol is mixed with water to form a 70% solution suitable for use as an ingredient in forming compositions in accordance with the present disclosure. In some embodiments, a 70% sorbitol solution may be added to the composition in an amount of about 0.1% to about 10.0% by weight of the total composition.

Preservatives such as antimicrobial preservatives may be used to prevent or inhibit the growth of micro-organism which could present a risk of infection to the user or degrade the vitamin C. Thus, suitable antimicrobial preservatives include ingredients capable of retarding the oxidation of vitamin C and/or extending the shelf-life of active ingredients. The properties of these antimicrobial substances typically include chemical groups that are aggressive towards living cells. Non-limiting examples of suitable preservatives include quaternary ammonium salts, phenoxyethanol, amine salts, Na metabisulfite and combinations thereof. The antimicrobial preservatives may be present in an amount of about 0.1% to about 5.0% by weight of the total composition.

Suitable salts that may be employed in making stable vitamin C compositions in accordance with this disclosure include acid and base addition salts. Non-limiting illustrative examples of such acid salts include: inorganic acid addition salts such as hydrochloride, sulfate, and phosphate; and organic acid addition salts such as acetate, maleate, fumarate, tartrate, and citrate. Non-limiting examples of suitable basic salts include: metal salts such as the alkali metal salts such as the sodium salt and potassium salt; alkaline earth metal salts such as magnesium salt and calcium salt; and other salts such as aluminum salt, and zinc salt. Non-limiting examples of suitable ammonium salts are ammonium salt and tetramethylammonium salt. Non-limiting examples of suitable amine addition salts are salts with morpholine and piperidine. Non-limiting examples of suitable amino acid addition salts include salts with lysine, glycine, and phenylalanine. The one or more salts may be present in an amount of about 0.01% to about 4.0% by weight of the total composition. In embodiments mixtures of salts have been found to further promote stability, especially when combined with a reducing sugar such as sorbitol. In one embodiment, Ca Hydroxide (in an amount of about 0.01-0.5% by weight of the total composition) is combined with Zn Chloride (in an amount of about 0.01-2.0% by weight of the total composition). The combination of salt admixtures with reducing sugar was found to promote stability of aqueous ascorbyl acid solutions.

In embodiments, alkaline earth metal salts such as magnesium and calcium salts may be provided in a unique stability promoting admixture. It is believed that the combination of such metal ions in solution promotes the stability of the compositions. Other salt mixtures such as zinc salts and aluminum salts also promote stability.

In embodiments, the stable compositions may optionally include surfactants. Suitable surfactants for use with the compositions of the present disclosure include ionic or nonionic surfactants, used alone or in admixture. Non-limiting examples of suitable surfactants include alkyldimethylbenzylamines, cetearyl alcohol and sodium cetearyl sulfate, PEG-1000 monocetyl ether, quaternary ammonium salts such as alkyl trimethyl ammonium bromide, polyol ester glycerol monostearate and potassium stearate, sodium lauryl sulfate (SLS), ethoxylated fatty alcohols, and/or combinations of these surfactants. Fatty acids like stearic acids may be included to regulate the consistency of the composition. Optionally, polymers such as carbomers can be included in the present compositions. Particularly useful surfactants for use in the aqueous phase are sodium lauryl sulfate, saponins or combinations thereof. The surfactants may be present in an amount of about 0.01% to about 20% by weight of the total composition. In embodiments, the surfactants are present in an amount of about 0.1% to about 5% by weight of the total composition.

The inclusion of various surfactants in compositions of the present disclosure increases the percutaneous absorption of vitamin C when solutions are applied to skin. It is believed that by adding surfactants to the stable ascorbic acid solutions, the surface tension of the solution is decreased allowing for better absorption through skin. Thus methods of applying surfactant containing solutions to skin in order to increase the levels of vitamin C absorbed into the skin are also described herein. Accordingly, surfactant may also be included in the compositions of the present disclosure in amounts sufficient to increase the absorption of vitamin C through skin. For example, by adding sodium lauryl sulfate (SLS) (e.g., as a 30% solution) in an amount of about 0.1% to about 5% by weight of the total composition, and then applying the solution to skin, the percutaneous absorption of vitamin C through skin is increased in comparison to vitamin C solutions that do not have surfactant.

In embodiments, the compositions in accordance with the present disclosure may optionally include alcohol. Non-limiting examples of suitable alcohols include lower aliphatic alcohols such as methanol, ethanol, propanol, and other lower alcohols, used alone or in admixture. One particularly useful alcohol for use in the aqueous vitamin C compositions in accordance with the present disclosure is ethanol. Alcohol may be present in an amount of about 0.01% to about 20% by weight of the total composition. In embodiments, the alcohol is present in an amount of about 0.1% to about 5% by weight of the total composition. Without wishing to be bound by any theory, it is believed that when alcohol is added to the aqueous vitamin C compositions in accordance with the present disclosure a process of transesterification stabilizes the negative charge of the ascorbate anion and slows down the process of decarboxylation. Accordingly, the addition of alcohol, polyols, as well as any other chemical that slows down the decarboxylation of the unstable ascorbate anion promotes the stability of the aqueous compositions in accordance with the present disclosure.

The pH of the aqueous compositions in accordance with the present disclosure may be adjusted to be about 2 to about 6, and, in some particularly useful embodiments below 5. The pH of the composition ensures that most of the ascorbic acid remains in the protonated, uncharged form. The protonated form of ascorbic acid used in compositions of the present disclosure is believed to remove the ionic repulsion of the two oxygen groups, thus helping to stabilize the molecule. Also because the protonated form of ascorbic acid is uncharged, entry into the skin (which itself has a pH of about 3-5) is believed to be facilitated. Agents suitable for adjusting the pH of the aqueous phase include, but are not limited to citric acid, phosphoric acid, lactic acid or glycolic acid. The pH adjustment agents may be present in an amount of about 0.01% to about 5% by weight of the total composition. In embodiments, the pH adjustment agent is present in an amount of about 0.1% to about 1.0% by weight of the total composition.

In embodiments, the stable compositions in accordance with the present disclosure may include secondary reducing agents. Non-limiting examples of suitable secondary reducing agents include propyl gallate and sulfites, including sulfites, bisulfites, metabisulfites, their salts, and their derivatives. In one particular embodiment, sodium metabisulfite may be added as a secondary reducing agent. Since vitamin C has a tendency to oxidize, antioxidants may be advantageous because they have greater tendencies to oxidize than vitamin C. Sodium metabisulfite has the added advantage that it does not discolor by oxidation. In vitamin C and sodium metabisulfite compositions, it is believed that the sodium metabisulfite oxidizes first and delays the start of any oxidation of the vitamin C, so that excessive oxidation is delayed or totally avoided. The secondary reducing agent may be present in an amount of about 0.1 to about 10% by weight of the total composition. In some embodiments, the reducing agent is present in an amount of about 0.5% to about 5% by weight of the total composition.

Other suitable optional ingredients include moisturizing agents (such as Na Hyalurate solution) and fragrance. Na hyalurate moisturizing agent that is synonymous with and refers to hyaluronic acid, sodium salt; sodium hyaluronate; hyaluronic acid; or sodium hyalurate and has the general formula (C₁₄H₂₀NO₁₁Na)ₙ. Na hyalurate 1% solution may be present, for example, in an amount of about 0.001 to about 0.2% by weight of the total composition, or in amounts that effectively moisturize the formulations.

The viscosity of the final vitamin C composition can be in an amount of about 30 to 10,000 centipoise (cps), in embodiments about 30 cps and about 250 cps. The specific gravity of the final composition can be in an amount of about 1.00 and 1.15, in embodiments, about 1.02 and about 1.06.

In embodiments, the vitamin C compositions in accordance with the present disclosure may be a substantially clear, viscous liquid to a semi-viscous lotion. Those skilled in the art will envision testing to confirm the shelf life of the products described herein. Further testing methodology is described below.

The aqueous compositions in accordance with the present disclosure can be prepared by mixing the various ingredients while mixing and heating to 70-75° C.

In embodiments, patients are treated by topically applying to skin in need of vitamin C one or more compositions including vitamin C, water, and one or more reducing sugars. In some embodiments, the composition may further include surfactant, secondary reducing agents, alcohol, and other ingredients as described herein. The vitamin C is applied until the treatment goals are obtained. However, the duration of the treatment can vary depending on the severity of the skin condition. For example, treatments can last several weeks to months depending on the goal of treatment. In treatment embodiments, 1 to 5 drops of a composition containing vitamin C may be applied to skin twice a day for 4 weeks.

In embodiments, the aqueous vitamin C compositions are applied for cosmetic purposes only.

In some embodiments, use of vitamin C compositions as described herein may be included in the manufacture of a medicament for treatment of a skin condition. In such embodiments, vitamin C described in accordance with the present disclosure can be manufactured into a pure medicament, compositions containing medicament, and/or formulations containing medicament and any excipients and/or ingredients described herein.

The following non-limiting examples further illustrate compositions and methods in accordance with this disclosure.

### EXAMPLE 1

Example 1 below shows a non-limiting example of a suitable composition in accordance with the present disclosure.

| | Weight percent of the total |
|---|---|
| Ingredient | composition |
| Water | 60-94% |
| Sorbitol 70% | 0.1-10% |
| Ca Hydroxide | 0.10-0.5% |
| Zn Chloride | 0.10-2% |
| Na Hyalurate | 0.001-0.02% |
| Ascorbic acid | 5-40% |
| SLS (30% solution) | 0-5% |
| Phenoxyethanol | 0.1-1% |
| Fragrance | 0.0-5% |
| Alkydimethylbenzylamine | 0-5% |

### EXAMPLE 2

Example 2 below shows another suitable non-limiting example of a composition in accordance with the present disclosure.

| | Weight percent of the total |
|---|---|
| Ingredient | composition |
| Water | 60-94% |
| Reducing Sugar | 0.1-10% |
| Metal Salt or mixtures thereof | 0.10-5% |
| Moisturizing Agent | 0.0-0.02% |
| Ascorbic acid | 5-40% |
| Antimicrobial | 0.0-1% |
| Surfactant | 0.0-5% |
| Fragrance | 0.0-5% |
| Alcohol | 0.0-20% |

The compositions of the present disclosure may be packaged in suitable containers such as tubes or bottles. Suitable containers are commercially available from a variety of suppliers. A wide variety of containers and suppliers are listed in the CPC Packaging Directory. (See, Buyers' Guide under “Containers” at www.cpcpkg.com). In embodiments, containers are selected with low oxygen permeability. Suitable containers include containers made from high density polyethylene and the like.

### EXAMPLE 3

Stability Study

In vitamin C compositions without reducing sugar, an aqueous solution of 5% ascorbic acid will likely decompose to less than 90% of the concentration at room temperature in 4 weeks time. See, for example, U.S. Pat. No. 4,983,382 the entire disclosure of which is incorporated herein by this reference.

Conversely, the stability of compositions made in accordance with the present disclosure show improved stability. Such compositions were evaluated by placing aliquots of each example in an oven at 5, 25, 30 and 40 degrees Centigrade for predetermined time periods and at the end of each time period analyzing the amount of vitamin C present in the composition.

The following results were observed with compositions in accordance with example 1 having 20% initial vitamin C concentration, sorbitol 70%, Ca hydroxide, Zn chloride, Na hyaluronate 1%, SLS (30% solution), phenoxyethanol and fragrance.

| Stability of formulation of example No. 1 | % Vitamin C |
|---|---|
| Initial amount of Vit. C | 20% |
| 6 weeks at 40° C.: | 16.52% |
| 3 months at 25° C.: | 17.50% |
| 3 months at 40° C.: | 13.23% |
| 8 months at 25° C. | 16% |

Conversely, vitamin C composition without reducing sugar showed only 9.7% vitamin C remaining after 3 months at 40° C.

### EXAMPLE 4

Percutaneous Absorption Study:

The in-vitro percutaneous absorption of vitamin C formulations were compared using intact human cadaver skin. Cumulative transdermal absorption of radiolabeled [¹⁴C] L-ascorbic acid was measured at 24 hours. The human cadaver skin was obtained from a single donor and dermatomed to approximately 500 micron thickness. The skin samples were mounted on Franz static diffusion glass chambers. The skin surfaces of approximately 1.77 cm² were washed with 0.5 ml of water at 37° C. for 10 seconds. The water was aspirated and the surface pad dried. The following treatments were performed.

Treatment A. 15 mg of first formulation in accordance with example 1 having 20% ascorbic acid, reducing sugar, metal ions, reducing agent, and anionic surfactant (1% SLS) was applied to 1.77 cm² of human cadaver skin samples.

Treatment B. 15 mg of first formulation in accordance with example 1 having 20% ascorbic acid, and metal ions was applied to 1.77 cm² of human cadaver skin samples. This solution did not contain anionic surfactant.

Table A summarizes the skin absorption results observed.

TABLE A

AMOUNT OF ASCORBIC ACID
ABSORBED IN 24 HOURS

| Treatment | In micrograms | as % of amount applied |
|---|---|---|
| A | 403 | 13.35 |
| B | 313 | 10.00 |

The foregoing data demonstrates that the addition of a surfactant to stable formulations in accordance with the present disclosure increases absorption of vitamin C. For example, the addition of 1% SLS increased the percutaneous absorption of vitamin C by approximately 30%.

### EXAMPLE 5

Another non-limiting example of a composition in accordance with the present disclosure is prepared by combining the following three phases A, B, and C:

| | | Weight percent of the | Weight for 1000 | |
|---|---|---|---|---|
| Phase | Ingredient | total composition | Kg batch | |
| A | Distilled Water | 60-70 | 600-700 | Kg |
| A | Sorbitol 70% Solution | 0.5-5% | 5-50 | Kg |
| A | Zinc Chloride | 0.005-1% | 0.05-10 | Kg |
| A | Calcium Hydroxide | 0.005-1% | 0.05-10 | Kg |
| A | Sodium Hyaluronate | 0.5-5% | 5-50 | Kg |
| | 1% Solution | | | |
| A | Ascorbic Acid | 15-20% | 150-200 | Kg |
| A | Ethyl Alcohol (200 | 10-20% | 100-200 | Kg |
| | Proof SDA 39C) | | | |
| A | Sodium Lauryl Sulfate | 0.05%-5% | 0.5-50 | Kg |
| B | Phenoxyethanol | 0.5-5% | 5-50 | Kg |
| C | Green Tea/Vit. E ext | 0.0-1% | 0-10 | KG |
| C | Fragrance | 0.00-1% | 0-10 | Kg |

Phase A is made by adding water to a container and heating to 55° C. while mixing. Each Phase A ingredient (except Vitamin C, Ethyl Alcohol and SLS) is added to the water and mixed into solution before adding the next ingredient. At 55° C. vitamin C is added under continuous mixing until dispersed. Next, ethyl alcohol is added. While maintaining the temperature of 55° C. the Phase A ingredients are mixed for 5 minutes. Next, the temperature is lowered to 30-40° C., and SLS in added and mixed until dissolved.

Next, the Phase A mixture is heated to a temperature of 45-50° C. and the Phase B ingredient (Phenoxyethanol) is added to form an admixture.

Next, the Phase A, Phase B admixture is cooled to 35-40° C. and the Phase C ingredients (fragrances) are added. The finished product is placed in containers.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

What is claimed is:

1. A composition comprising:
vitamin C;
water; and
a reducing sugar.

2. A composition according to claim 1, wherein the reducing sugar is selected from the group consisting of mannitol, sorbitol, xylitol, maltitol, lactitol, and combinations thereof.

3. A composition according to claim 1, wherein the reducing sugar is present in an amount of about 0.1% to about 10% by weight of the total composition.

4. A composition according to claim 1 wherein the vitamin C is present in an amount of about 5.0% to about 40% by weight of the total composition.

5. A composition according to claim 1, wherein the water is present in an amount of about 40% to about 96.0% by weight of the total composition.

6. A composition according to claim 1, wherein water is present in an amount greater than 40% by weight of the total composition and vitamin C is present in an amount greater than 15% by weight of the total composition.

7. A composition according to claim 1, wherein the pH is about 2 to about 6.

8. A composition according to claim 1, wherein the pH is below 5.

9. A composition according to claim 1 comprising a salt selected from the group consisting of acid addition salt, base addition salt, metal salts, alkali metal salts, alkaline earth metal salts, ammonium salts, amine addition salts, amino acid addition salts, and combinations thereof.

10. A composition according to claim 9, wherein the salt is present in an amount of about 0.01% to about 4.0% by weight of the total composition.

11. A composition according to claim 9 wherein the salt comprises calcium hydroxide, zinc chloride and combinations thereof.

12. A composition according to claim 1, comprising surfactant.

13. A composition according to claim 12 wherein the surfactant is present in an amount of about 0.01% to about 20% by weight of the total composition.

14. A composition according to claim 12, wherein the surfactant comprises sodium lauryl sulfate, cetearyl alcohol, sodium cetearyl sulfate, PEG-1000 monocetyl ether, quaternary ammonium salt, polyol ester glycerol monostearate, potassium stearate, ethoxylated fatty alcohols, fatty acids, saponins, carbomers, or combinations thereof.

15. A composition according to claim 1 comprising a secondary reducing agent.

16. A composition according to claim 1 wherein the secondary reducing agent is present in an amount of about 0.1% to about 10% by weight of the total composition.

17. A composition according to claim 15, wherein the secondary reducing agent is selected from the group consisting of bisulfites, metabisulfites and salts thereof, sodium metabisulfite, propyl gallate and sulfites, and combinations thereof.

18. A composition according to claim 1 comprising a preservative.

19. A composition according to claim 18, wherein the preservative is an antimicrobial preservative selected from the group consisting of amine salts, Na metabisulfite, phenoxyethanol, quaternary ammonium salts, and combinations thereof.

20. A composition according to claim 1 comprising alcohol.

21. A composition according to claim 20, wherein the alcohol is present in an amount of about 0.01% to about 20% by weight of the total composition.

22. A method of increasing absorption of vitamin C in skin comprising:
topically applying a composition comprising vitamin C, water, surfactant, and a reducing sugar.

23. A method according to claim 22 wherein the surfactant is anionic.

24. A method according to claim 22 wherein the surfactant is sodium lauryl sulfate.

25. A method according to claim 22 wherein the surfactant is present in an amount of about 0.1% to about 20% by weight of the total composition.

26. A method according to 22 wherein the surfactant is present in an amount of about 0.1% to about 5.0% by weight of the total composition.

27. A method according to 22 wherein the water is present in an amount greater than 40% by weight of the total composition and vitamin C is present in an amount greater than 15% by weight of the total composition.

28. A composition comprising:
vitamin C in an amount greater than 15% by weight of the total composition;
a salt selected from the group consisting of acid addition salt, base addition salt, metal salts, alkali metal salts, alkaline earth metal salts, ammonium salts, amine addition salts, amino acid addition salts, and combinations thereof,
water in an amount greater than 40% by weight of the total composition;
a secondary reducing agent; and
reducing sugar selected from the group consisting of mannitol, sorbitol, xylitol, maltitol, lactitol, and combinations thereof.

29. A method of improving the appearance of skin comprising the steps of:
(a) stabilizing ascorbic acid in a solution which comprises water, a reducing sugar, and salt wherein at least about 0.1% of the total weight of said cosmetic solution is reducing sugar; and
(b) topically applying said solution to the area of skin to be affected such that said ascorbic acid is absorbed by said skin.

30. A method according to claim 29 wherein said solution comprises the composition of claim 1.

31. A method according to claim 29 wherein the solution comprises surfactant.

32. A method according to claim 29 wherein the solution comprises alcohol.
